(19) United States
(12) Patent Application Publication
(10) Pub. No.: US 2004/0101570 A1
(43) Pub. Date: May 27, 2004

(54) Animal bone fiber and method of producing the same

(76) Inventor: Sanai Fujita, Tokorozawa-shi (JP)

Correspondence Address: NILS H. LJUNGMAN & ASSOCIATES, P. O. BOX 130, GREENSBURG, PA, 15601-0130, US

(21) Appl. No.: 10/304,714

(22) Filed: Nov. 26, 2002

(51) Int. Cl. A61K 35/32; B32B 5/02

(52) U.S. Cl. 424/549; 442/123; 55/522

(57) ABSTRACT

The bones of animals such as bovines and horses are, for example, boiled, dried sufficiently, melted under heating and made into a fibrous or filamentous form to produce animal bone fiber using animal bones as raw material.

These animal bones have various effects such as an alkalinizing effect, antibacterial ability and adsorbing ability. The specific surface area of these animal bones can be increased if they are made into a fibrous or filamentous form and therefore, various effects can be produced more efficiently. Also, these bones can be used for a wider application by utilizing known fiber technologies.

### BACKGROUND OF THE INVENTION

The present invention relates to a new use for the bones of animals such as bovines and horses.

Formerly, almost of the bones of animals such as bovines and horses were disposed of as waste. In recent years, these animal bones have been found to have various working effects and the utilization of these animal bones are started in various uses at present.

For example, in Japanese Patent Application Publication (JP-B) No. 6-61460 discloses a material obtained by mixing and burning burnt bone powder and clayey material has an alkalinizing effect, and also, produces a freshness-retaining function due to prevention of oxidation and a filtrate coagulating effect. By making use of these effects, activators are utilized effectively as materials for retaining the freshness of foods, as filter materials for waste liquids and used cooking oil, and further, as coagulants for organic materials.

Japanese Patent Application Laid-Open (JP-A) No. 8-133902 discloses freshness-retaining packaging materials formed by impregnating, coating or mixing a part of a sheet material using, as a base material, a non-woven fabric, synthetic resin or rubber with burnt powder of animal bone, namely, freshness-retaining packaging materials having various functions such as anti-oxidation ability, bactericidal ability and mildewproof ability.

JP-A Nos. 8-117002, 8-135042 and 8-134774 focus on the deodorant characteristics, drying effect and antibacterial ability of burnt powder of animal bone and disclose deodorant drying sheet materials, insoles of shoes, clothes and bedding covers, which are obtained by impregnating, coating or mixing a thin layer material with burnt powder of animal bones.

JP-A No. 8-243121 focuses on the adsorbing ability of burnt powder of animal bone and discloses a sheet material which surely absorbs and coagulates liquid excretions and prevents these excretions from leaking externally, removes an offensive odor generated from these excretions, disinfects generated bacteria and further can keep the surface portion in a dry condition.

Also, JP-A No. 9-59137 discloses dentifrice produced using a 250 to 350-mesh bone powder of animal bones as an effective component.

### SUMMARY OF THE INVENTION

This invention relates to animal bone fiber made from animal bone and method of producing the same.

It is an object of the present invention to draw out the nature, which animal bones possess, more efficiently and to spread the use of animal bones more widely.

The present invention provides animal bone fiber made from animal bone that is not a material formed by impregnating, coating or mixing fiber, but a material obtained by heat melting animal bones and forming fiber from the molten animal bones.

Bones of animals, such as bovines and horses, have various effects, such as an alkalinizing effect, antibacterial ability and adsorbing ability. The animal bones are used as raw material to form fiber, whereby the said various' effects can be produced more efficiently. Also, because known technologies, such as fiber technologies for producing various yarns, woven and non-woven fabrics, and various products utilizing these fiber materials from fiber bodies, can be utilized, the range of use for utilizing these animal bones can be spread more widely. As a consequence, animal bones, such as bovine bones, can be effectively utilized in industrial fields.

### DETAILED DESCRIPTION

The present invention provides animal bone fiber made from animal bone obtained by heat melting and making animal bone into a form of fiber or filament.

To be more specific, the present invention provides fiber made from animal bone obtained by boiling animal bones, then, if necessary, drying the bones sufficiently, and melting under heating to make into a fiber or filament form.

As aforementioned, animal bones have various effects, such as an alkalinizing effect, antibacterial ability and adsorbing ability. If fibrous or filamentous bodies are formed from animal bones by heat melting, the said various effects can be produced more efficiently since the said bodies have a larger surface area. Further, because the fibrous or filamentous bodies can be made into various forms such as non-woven fabrics and fiber clothes, the use of animal bones can be spread more widely. These fibrous or filamentous bodies may be used to form, for example, clothes, such as underwear and socks, which combine both antibacterial ability and deodorant ability; antibacterial cloth bodies, such as handkerchiefs, towels and disinfecting clothes, which combine both antibacterial ability and deodorant ability; bedding sheet bodies such as domestic or hospital bed covers, sheets, pillow covers and blankets made of toweling; gas filters such as air filters used in air conditioners, air cleaners and the like; and liquid filters, such as filter sheets for waste water, waste oil and the like.

The present invention also provides a method of producing animal bone fiber, comprising boiling, drying, and heat melting animal bone to form fiber from the molten bone. In this case, an appropriate method being able to remove organic materials and the like, adhering to the outside of the bone and to the inside of pores of the bone, may be adopted as a method used in place of the boiling.

As to the fiber forming of animal bone fiber, for example, animal bones are melted in a melting kettle after organic materials and the like adhered to the outside of the bone and to the inside of pores of the bone as aforementioned are removed, and then, the molten bone can be formed into fiber by a known fiber-forming method, such as a method winding the molten materials forced out from nozzles, a method winding the molten bone which is made into thin form by flinging away with centrifugal forces, and flame spraying method.

The method of producing animal born fiber according to the present invention will be hereinafter explained in more detail.

As aforementioned, the animal bone fiber in the present invention can be produced by boiling animal bone, drying sufficiently followed by heat melting to obtain molten animal bone, and making the said molten bone into a form of fiber or filament.

As the “animal bone” as raw material, one or two, or more types selected from the bones of organisms such as the mammals, fish, birds and others, shells and corals may be used. However, bones primarily including the bones of herbivorous animals such as bovines, horses and sheep, and particularly, raw bones dumped as waste from stud farms, cattle farms and other farms are preferable. It is to be noted that pigs and wild boars almost have cartilaginous bones and a large part of these bones probably solve in a boiling step during a series of the production process. Therefore the bones of bovines, horses and sheep are preferable.

The “animal bone” as raw material is preferably cut or crushed into an appropriate size (for example, 5 to 50 mm, preferably 5 to 30 mm) to make born grains or bone chips, which are then boiled In this case, when the bone is made into an excessively fine powder form, the melting rate of the bone material is made very low by thermo-pressure.

The cutting or crushing of the bone material may be carried out in the following manner. Specifically, the bone material after boiled is frozen once in a freezer (freezing chamber) or the like and the frozen bone is crushed. This is because the bone after boiled is easily frozen since a lot of water remains in the pores of the bone and the frozen bone is easily crushed. The temperature of the bone when frozen is preferably about −100 to −40° C. and the freezing time is preferably about 1 to 3 hours.

The boiling is a process of separating and removing organic materials such as glue, enamel, fat and bone marrow, and makes it possible to separate and remove organic materials adhering not only to the outside of the bone but also to the inside of the pores.

Specifically, in the boiling step, the bone grain or the bone chip is poured into an oven (compression kettle) and boiled at about 200 to 400° C. for 60 minutes under a pressure of about 5 tam. In this case, if caustic soda or a finished bone powder is mixed in the oven when the bone material is boiled, the organic materials are specified by the action of an alkali such as caustic soda or potassium hydroxide, so that they are easily dissolved in water. Therefore, the effect of separating the organic materials is promoted and the organic materials are thereby easily removed whereby the time required for boiling the bone material can be shortened. The amount of caustic soda or finished bone powder to be mixed is preferably about 200 to 300 cc in the case of caustic soda, and about 500 g to lkg in the case of the finished bone powder, on the basis of 200 kg of the raw bone.

It is preferable that water or hot water be poured on the bone after boiled, and then, dried thoroughly. At this time, the organic materials and other soils can be washed off more perfectly by treating the bone with caustic soda.

The animal bone grain or chip obtained in this manner is melted by heating it to about 1270 to 2000° C. and preferably about 1400 to 1800° C. in a melting kettle. The molten bone is flung away by centrifugal force to make it thin, and further, a high-speed air stream is applied to the molten bone to make the bone into fiber, which is then wound around a rotating drum, and thus animal bone fiber can be formed.

As to the method of forming fiber, for example, one method that the molten bone material is forced out from a nozzle and wound is applicable. Another method is that the molten bone material is fed to the vicinity of the center of a rotary plate and developed into a liquid film on the rotary plate with centrifugal force. The liquid bone film is scattered from the periphery of the rotary plate in a droplet manner, allowed to flow down along the inside wall of the equipment and discharged from the outlet port to the outside of the system. Then, the liquid bone film is fed to a spinner through a gear pump and then spun there whereby the bone can be made into fiber. The rotary plate used in this case is preferably kept at a high temperature to improve the fluidity of the molten bone material.

In the above production process, the boiled bone material may be burnt at about 900 to 1100° C. before heat melting. In the case where, particularly, the organic materials and the like can be removed insufficiently by boiling or other means, these materials can be removed by burning.

This animal bone fiber of the above process is made into any of fiber clothes, non-woven fabrics, block bodies and other forms. From any of these fiber materials, the followings may be formed, such as: clothes such as underwear and socks, antibacterial cloth bodies such as handkerchiefs, towels and disinfecting clothes, bedding sheet bodies such as domestic or hospital bed covers, sheets, pillow covers and blankets made of toweling, gas filters such as air filters used in air conditioners, air cleaners and the like and liquid filters such as filter sheets for waste water, waste oil and the like.

Another example is that, a binder is sprayed on the animal bone fiber to collect it as a fiber flocculate on a collecting conveyer, the binder is cured to make a mat body, which is then fed to a de-weaving machine to de-weave the mat body into small flocculate groups having an irregular form. These flocculated materials may be used as, for example, insulation materials that are provided with antibacterial ability and deodorant ability, and are to be packed in a space under the roof or floor.

Also, the animal bone fiber is woven to form a sheet, from which bedding sheet bodies such as domestic or hospital bed covers, sheets, pillow covers and blankets made of toweling may be formed.

In addition, woven or non-woven fabric is formed from the animal bone fiber and from the woven or non-woven fabric, gas filters such as air filters used in air conditioners, air cleaners and the like and liquid filters for waste water, waste oil and the like may be formed.

What is claimed is:

1. An animal bone fiber formed by making the heat-melting animal bone into a fibrous or filamentous body.

2. The animal bone fiber according to claim 1, the fiber being formed by making the animal bone into a fibrous or filamentous body by boiling the animal bone and then melting it under heat.

3. The animal bone fiber according to claim 1, wherein the animal bone primarily comprises the hard bone of herbivorous animals such as bovines, horses and sheep.

4. An antibacterial cloth body including a handkerchief, a towel and a disinfecting cloth, the antibacterial cloth comprising the animal bone fiber according to claim 1.

5. A bedding sheet body comprising the animal bone fiber according to claim 1.

6. A gas filter comprising the animal bone fiber according to claim 1.

7. A liquid filter sheet material comprising the animal bone fiber according to claim 1.

8. A method of producing an animal bone fiber, comprising boiling an animal bone, drying the boiled bone, melting the dried born under heating and winding the molten bone.

9. The method of producing an animal bone fiber according to claim 8, wherein the animal bone primarily comprises the hard bone of herbivorous animals such as bovines, horses and sheep.
